(19) United States
(12) Patent Application Publication
(10) Pub. No.: US 2009/0240306 A1
(43) Pub. Date: Sep. 24, 2009

(54) Pancreatic stimulator for type II diabetes

(76) Inventor: Harish K. Kapoor, Parkland, FL (US)

Correspondence Address: Harish K. Kapoor, 6051 N. W. 60th. Ave., Parkland, FL, 33067, US

(21) Appl. No.: 12/382,444

(22) Filed: Mar. 17, 2009

Related U.S. Application Data
(60) Provisional application No. 61/069,882, filed on Mar. 19, 2008.
(60) Provisional application No. 61/132,451, filed on Jun. 19, 2008.

(51) Int. Cl. A61N 1/00

(52) U.S. Cl. 607/50

(57) ABSTRACT

The present invention relates to methods of preventing, managing and/or treating blood sugar disorders, particularly those related to diabetes, by stimulating pancreas externally and non-invasively. Therapeutically repairing and regenerating beta cells of islets of langerhans enhance their ability to produce insulin to treat high blood sugar. The transformation of insensitive or impaired islets of langerhans results in an increased insulin secretion and in increased insulin level in blood plasma. This improves the glucose toxicity and improves beta cells dysfunction. This also regulates the pancreatic secretion that induces a feeling of satiety, which leads to weight loss.

### CROSS-REFERENCE TO RELATED APPLICATION

This is a non-provisional application of provisional application No. 61/069,882 filed on Mar. 19, 2008, and entitled “Pancreatic Stimulator for type II diabetes.”

This application makes reference to U.S. Provisional Patent Application Ser. No. 61/132,451, filed Jun. 19, 2008, and entitled “Dedicated Diabetic Machine (DDM).”

### FIELD OF INVENTION

The present invention relates for preventing, managing or treating diabetes and obesity, by stimulating pancreas externally and non-invasively.

### BACKGROUND

About 97 million Americans are obese and one third of American kids are destined to be diabetic. Based on the CDC predictions, 45 million to 50 million Americans could have diabetes by 2050. Consumer's recent frustration from prescription drugs has strengthened their belief in therapeutically techniques and alternate medicine. This frustration is also coupled with long term dangerous side effects from use of pharmaceutical drugs. Benefit from the proposed device outweighs the risk from modern medicine.

The pancreatic stimulator will therapeutically treat diabetic patients, to increase the relative insulin producing performance of endogenous pancreatic beta cells into insulin producing beta cells, and also regulate pancreatic secretion leading to weight loss.

Obesity and overweight are risk factors for type 2 diabetes, hypertension and coronary heart disease that cause morbidity, mortality and high health-care expenditure.

Obesity is the number one problem in the U.S. An estimated one third of Americans are overweight, with an additional 25 percent being classified as obese. Being overweight significantly increases a person's risk of developing diabetes, heart disease, stroke

Type 2 diabetes is a chronic disease associated with high rates of morbidity and premature mortality. An alarming increase in the prevalence of type 2 diabetes is expected and the need for preventive action is widely acknowledged. While increased physical activity and restriction of energy intake can substantially reduce the incidence of type 2 diabetes, insight into the role of other lifestyle factors may contribute to additional prevention strategies for type 2 diabetes.

Diabetes is a metabolic abnormality mainly of glycometabolism, resulting from insufficient insulin secretion, decreased sensitivity of target cells of insulin and so forth, and principally characterized by noticeable hyperglycemia. If the hyperglycemia continues for a long period of time, serious complications arise in various organs and nerves such as retinopathy, nephropathy and neuropathy, which are caused mainly by vascular lesion. Therefore, for the treatment of diabetes, it is extremely important to control and maintain blood glucose level at a normal level, and methods for that purpose have been studied since old days.

The objective of the present invention is to provide a focused approach to control weight which can increase metabolism, and control diabetes mellitus. As a result the present invention strives to give full spectrum diet and physical activity.

### SUMMARY OF THE INVENTION

The pancreatic stimulator therapeutically treats obese and diabetic patients, to increase the relative insulin producing performance of endogenous pancreatic beta cells.

The techniques and methods will stimulate pancreatic acini and islets of langerhans primarily the Beta cells that block DPP4 enzyme and increase GLP-1 levels. DPP4 is essential for the control of GLP1 bioactivity and glucose homeostasis. DPP-4 is a key enzyme that is involved in the breakdown of a variety of gut peptides that possess a variety of activities. The intensity of the stimulus is calculated corresponding to the mass/unit area of the subject and is focused at the pancreatic anatomy.

For type II diabetes where onset is gradual and insulin therapy is not necessarily required for life support, blood glucose level can be controlled by combination of exercise therapy and drug therapy. As the drugs, insulin secretion enhancers, one of orally available hypoglycemic agents, have widely been used clinically. However, since currently available insulin secretion enhancers all promote insulin secretion non-dependently on glucose level, they cause problems of severe hypoglycemia or insufficient control of blood glucose if doses are not appropriate, and are not fully satisfactory drugs. If a hypoglycemic agent or a therapy that is capable of enhancing insulin secretion dependently on a blood glucose level, the therapy can be extremely useful for blood glucose control of patients suffering from diabetes because the risk of hypoglycemia due to an excess dosage can be avoided.

According to other aspects of the present invention, pancreatic stimulation is a method for the therapeutic treating diabetes which comprises of administering a focused stimulation directly to beta cells.

This invention relates to a method for diabetes management and more particularly to therapeutic device for determining focused stimulation based upon the content of carbohydrates in foods ingested by a user.

### DETAILED DESCRIPTION

Two incretins—glucose-dependent insulinotropic polypeptide (GIP) and glucagon-like peptide-1 (GLP1)—potentiate glucose-dependent insulin secretion from islet-cells by activating specific G-protein-coupled receptors1. GLP1 also inhibits glucagon secretion and gastric emptying and induces a feeling of satiety, which leads to weight loss. To reduce the weight of the obese adult patient, reduce the caloric intake of the obese adult patient, or inhibit insulin hypersecretion by pancreatic beta-cells of the obese adult patient

Diabetes is one of typical diseases incident to old adults, similarly to cancers, brain infarction, cardiac infarction, Alzheimer's disease and the like. Diabetes is diseases caused by occurrence of abnormal metabolisms of glucose, protein and lipid due to a deficiency or insufficiency of the actions of insulin. Typical signs of diabetes include an abnormal increase in the serum glucose level over the normal range of the glucose level and an excretion of glucose in the urine.

Diabetes mellitus is a disorder of carbohydrate metabolism resulting from insufficient production of or reduced sensitivity to insulin. In persons who have diabetes, the normal ability of body cells to use glucose is inhibited, thereby increasing blood sugar levels. As more glucose accumulates in the blood, excess levels of sugar are excreted in the urine. Corresponding symptoms of diabetes include increased urinary volume and frequency, thirst, itching, hunger, weight loss, and weakness.

Type I is insulin-dependent diabetes mellitus for which insulin injection is required. In this type, insulin is not secreted by the pancreas and must be taken by injection. Type II, non-insulin-dependent diabetes mellitus may be controlled by dietary restriction. It derives from sluggish pancreatic secreted insulin.

The extended presence of GLP-1, in particular in the pancreatic tissue facilitates differentiation and regeneration of the beta cells already present that are in need of repair. These repaired insulin producing cells can contribute to the correction and maintenance of normal physiological glycemic levels.

Complications from diabetes typically involve the cardiovascular system which accounts for the majority of diabetes-related deaths. Other serious complications include a condition known as diabetic retinopathy (retinal changes leading to blindness), kidney disease, and frequent infection.

Medications have also been employed to help maintain blood glucose levels within target ranges. These medications improve the body's ability to use insulin and decrease the production of glucose by the liver. However, several of these medications have been taken off the market by the USFDA because of reported severe side effects. Other medications often cause nausea when used.

Insulin is one of hormones in pancreas and has such functions to promote the permeability of glucose through the cell membranes in liver, muscles and tissues and thereby to increase the uptake of glucose by the cells, and further to promote the combustion of glucose in the glycolysis step and oxidation step of glucose in the muscles and also to elevate the activity of the enzyme system for synthesizing glycogen from glucose. It is known that by exhibiting the above biological functions, insulin has such action “in vivo” as to keep the serum glucose level at the normal levels

In the management of diabetes it has been found that it is advantageous to monitor a patient's eating and exercise to determine the optimum insulin dosage required for that patient. With respect to monitoring a patient's eating it is extremely important to monitor the amount of carbohydrates which plays a critical role in determining the blood glucose level. Research has confirmed that if diabetic patients can control their blood glucose levels effectively they will reduce risk in developing many of the health complications well known in diabetes. There remains a need for a treatment that will improve the body's ability to use insulin and decrease the production of glucose released by the liver without adverse health effects. This is what this invention is targeting.

The present invention is directed to a method for treating diabetes. The treatment method of the present invention minimizes the complications from diabetes and helps to reverse many of its chronic side effects.

The subject invention offers advantages over existing treatment regimens for diabetic patients. By providing a means to stimulate the beta cells to regenerate, not only decrease the need for traditional drug therapy (Type 2) or insulin therapy (Type I). The maintenance of normal blood glucose levels also may reduce some of the more debilitating complications of diabetes

### Therapeutic Administration:

The process is accomplished by non invasive external electromechanical system where the interfacing transducers rest on the surface of the stomach in the close vicinity of pancreas. The device consists of (Ref. U.S. Provisional Patent Application Ser. No. 61/132,451 and entitled “Dedicated Diabetic Machine (DDM).”arrangement coupled with cam shaft to alternate the mechanical motion to transfer mechanical energy into beta cells. These repaired insulin producing cells can contribute to the maintenance of normal physiological glycemic levels.

The administration can also be accomplished in various other mechanical, electromechanical and hydraulically designed device or system to accomplish the said technique or method.

### EXAMPLE

The invention has been described with reference to exercise. Similarity have been drawn and document that pancreatic stimulation provides several times more concentrated effect than that provided by exercise. Because exercise acts like insulin! Exercise reduces blood glucose levels. There are many theories as to what happens, but in essence during exercise glucose is more effectively driven into the muscles and used as fuel. With continued moderate exercise muscles can take in 20 times the glucose they normally do. In addition exercise improves ongoing insulin sensitivity, promotes weight loss and helps remove fatty acids from the blood. As exercise builds more muscle mass it becomes a spiral of good getting better as there is more muscle to take in the glucose and less fat to impede the process. Exercise also improves blood flow, which is very important as many of the complications of diabetes are related to impaired blood flow in the extremities. Blood pressure is reduced, LDL (bad) cholesterol is reduced and HDL (good) cholesterol is increased. The multiplied concentrated input of pancreatic stimulation, corresponds to concentrated multiplied output from beta cells, that warrants that muscle can take 20 to 100 times glucose than they normally do. In other words the pancreatic stimulation can quadruple the benefit to a diabetic individual than driven from exercise.

### Relevant Literature

The practice of the Ayurveda and quantum medicine will help balance the endocrine system, tone the abdominal organs, stimulate both the nervous and Circulatory System, and reduce stress. Yoga also helps one to gain a better understanding of ones self, leading to acceptance and appreciation which will help eliminate the psychological reasons for Obesity. The practice of Yoga deals with all the aspects of an individual: the mind, body and spirit, giving a person control over his mind and body and making the effect is more permanent than other techniques

Clinical research in the west has focused exclusively on diabetes as a physical disorder. Clinical research in India, by contrast, has recognized that diabetes is a psychosomatic disorder, in which the causative factors are sedentary habits, physical, emotional and mental stress and strain. Most of these studies have recommended exercise of moderate intensity, as a means to adopt a regular diet and insulin dosage, or to control body weight and improve circulation.

Yoga addresses the whole person, considering not only their physical needs, but emotional, mental, intellectual and spiritual needs as well, through gentle movements, relaxation, breathing, lifestyle attitudes and meditation.

Many studies have shown the immediate effects of exercise on Type 2 diabetes. Exercise reduces the rate of blood glucose, increases the number of insulin receptors and increases the sensitivity and level of absorption of insulin by the tissues. Because of its hormonal and metabolic effects, it is believed that regular physical exercise can prevent or stop the development of Type 2 diabetes.

In summary, the regular practice of physical activities and exercise can have beneficial effects for diabetics. In the case of those with Type 2, often obese, exercise favors the loss of weight, increases the absorption of one's own insulin and diminishes the need for oral hypoglycemia. For persons with Type 1, exercise seems to bring little improvement to the metabolic control of diabetes, but reduces certain risk factors related to the heart. Diabetics must be aware of the possible problems which can arrive during or after exercise and know what to do about them.

Many studies have reported the beneficial effect of the practice of yoga on diabetes. Some studies have mentioned up to 65 percent beneficial effect of yogic therapy for diabetes. Regulate the diet throughout the program. Avoid simple sugars such as white sugar, honey, glucose and sweet eat complex carbohydrates such as wheat, oatmeal, buckwheat, corn, brown rice and beans. Avoid processed food and eat foods with lots of fiber and nutrients.

It has been concluded that the beneficial effects of the practice of yoga, is much more than a physical exercise. Whereas the affects of direct pancreatic stimulation are far better than exercise and yoga together. The pancreatic stimulation increases the blood supply, oxygen supply that in return improves efficiency and the functioning of pancreas along with all other benefits mentioned in the claims of this patent.

### REFERENCES

1. Zinman, B. Vranic, M. “Diabetes and exercise”, Medical Clinics of North America, January 1985, 69, 1: 145-157.
2. Shephard, R. S., “Physical activity and child health”, Sports Medicine, May/June 1984, 1: 205-233.
3. Campaigne, B. N. et al., “The effect of physical training on blood lipid profiles in adolescents with insulin-dependent diabetes mellitus”, The Physician and Sportsmedicine, December 1985, 13,12: 83-89.
4. Laporte, R. E. et all, “Pittsburgh insulin-dependent diabetes mellitus morbidity and mortality study: physical activity and diabetic complications” Pediatrics, December 1986, 78: 1027-1033.
5. Richter, E. A., Galbo, H., “Diabetes, insulin and exercise”, Sports Medicine, July/August 1986, 3,4: 275-288
6. Siscovick, D. S., Laporte, R. E., Newman, J. M. “The disease-specific benefits and risks of physical activity and exercise”, Public Health Report, March/April 1985, 100, 2: 180-188
7. Richter, E. A., Schneider, S. H., “Diabetes and exercise”, American Journal of Medicine”, 1981, 70: 201-209
8. Nadeau, Andre “L'activite physicque chez le diabetique”, Federation des medecins omnipraticens du Quebec, Congres “L'omnipraticien et le sport”, Quebec, 15 et 16 mai 1986, 12 p.
9. American College of Sports Medicine, “Guidelines for exercise testing and prescription, 3rd edition, Philadelphia, Lea and Febiger, 1986, 175 p.
10. Desai, B. P. “Influence of yogic treatment on serum lipase activity in diabetics”. Yoga Mimamsa Vol. XXIII, No. 3 & 4, p. 1 to 8, January 1985
11. Divekar, M. V. and Bhat “Effect of yoga therapy in diabetes and obesity”, Clinical diabetes update 1981, Diab. Assoc. India.
12. Koshti et al. “Electrophoretic pattern of serum proteins in diabetes mellitus as influenced by physical exercises (Yogasanas), Journal of the Mysore Medical Assoc. 36; p. 64, July 1972
13. Mohammad U. et al. “glucose tolerance and insulin therapy after yoga in diabetes mellitus”, Dept. of Medicine and Diabetology, Govt. Stanley Hospital, Madras (unpublished)
14. Patel C. H., “Yoga and Biofeedback in the management of hypertension”, The Lancet November 10, p. 1053-1055, 1973
15. Rugmini, P. S. and Sinha, R. N. “Effect of yoga therapy in Diabetes mellitus”, Seminar on yoga, Science and man, C.C., R.I. M.HI, p. 175-189, 1976
16. Sahay, B. K. et al. “The effect of yoga in Diabetes” in Bajay, J. S. “Diabetes mellitus in developing countries”, New Delhi, Interprint 1984, 379-381
17. Tulpule, T. H. “Yogic exercises and diabetes Mellitus (Madhumeh), Journal of Diab. Assoc. India Vol. 17, April 1977.
18. Udupa, K. N. “Stress and its management by yoga”, p. 305-32
19. Ramaiah, S. A. A., “Yoga Therapy for Diabetes: Washington, D.C. Study”, International Conference on Traditional Medicine, 1986, Madras. Published by Siddha Medical Board, Govt. of Tamil Nadu, Madras, India
20. Bhole, M. V. “Therapeutic applications of yoga techniques”, Yoga Mimamsa, Journal Kaivalyadhama, Vol. XXIII, No. 3 & 4, p. 29, October 1984
21. Gore, M. M. “Anatomy and Physiology of Yogic practices”, p. 74-96, Ed. 1985 by Kanchan Prakashan Kaivalyadhama, Lonavla.
22. Gore, M. M. “Yogic Treatment for Diabetes”, Yoga Mimamsa Vol. XXVI, no. 3& 4 pp. 130 to 145 October 1987, January 1988.
23. Bihar School of Yoga, Munger, Bihar, India under the direction of Dr. Shankardevananda MBBS
24. Vivekananda Kendra Yogas, Eknath Bhavan, no. 19, Gavipuram Circle, Bangalore, 560019, India. Tel. 0091-80-6597347 or 660.8645; fax: 0091-80-66-8645 or 667.3446 or 661.0666 email: vkyogas@bir.vsnl.net.in
25. The Yoga Institute, Prabhat Colony, Santacruz, (East) Bombay 400055
26. Kaivalyadhama Institute, Lonalva, India 410403 27. Shankardevananda, Dr. Swami, MBBS, “Yogic Management of Asthma and Diabetes”, Bihar School of Yoga”, Munger, Bihar, India pg. 76, 163-167
27. Bose, Buddha, Key to the Kingdom of Health, Calcutta, India, Statesman Press, 1938. Chopra, Deepak, M.D
28. Quantum Healing: Exploring the Frontiers of Mind/Body Medicine, New York, Bantam Books, 1989
29. Choudhury, Bikram, Bikram's Beginning Yoga Class, New York, Putnam Publishing Group, 1978
30. Funderburk, James, Ph.D., Science Studies Yoga, A Review of Physiological Data, Glenview, Ill., Himalayan International Institute of Yoga Science & Philosophy of USA, 1977
31. Jain, Suresh C., et al., “A Study of response pattern of non-insulin dependent diabetics to yoga therapy,” Diabetes Research and Clinical Practice, 1993, 19, 69-74
32. Mishra, S. K., “Diabetes mellitus in Indian medicine and its management by yoga,” Excerpta Medica ICS, 1979, 454, 373-378
33. Monro, Robin, et al., “Yoga Therapy for NIDDM: A controlled trial,” Complementary Medical Research, 1992, 6/1, 66-68
34 Ornish, Dean, M.D., Dr. Dean Ornish's Program for Reversing Heart Disease, New York, Ballantine Books.
35. Sinha, Phulgenda, Yogic Cure for Common Diseases, New Delhi, India, Orient Paperbacks, 1976. Julian Goldstein, B.S., M.

I claim:

1. Pancreatic stimulation is a method for preventing the development of pre-diabetes, diabetes and associated complications in an individual, wherein an effective amount of pancreatic stimulation is administered to said individual externally in need of such prevention.

2. Pancreatic stimulation is a method useful in regulating insulin resistance in obese that decrease the insulin resistance in said individual in need of such prevention.

3. Pancreatic stimulation is a method that regulates the pancreatic secretion that induces a feeling of satiety, which leads to weight loss.

4. Pancreatic stimulation is a method for therapeutically treating an individual exhibiting type II, insulin-resistant diabetes, effective in the reduction of blood glucose levels of a diabetic.

5. The method of claim 1, wherein the pancreatic stimulation therapy is applied to stimulate pancreas with truncated mechanical energy into beta cells.

6. The method of claim 1, wherein the pancreatic stimulation therapy is applied to stimulate a pancreas hydraulically.

7. The method of claim 3, wherein the pancreatic stimulation therapy is applied to stimulate the vagus nerve in the vicinity of pancreas.

8. The method of claim 3, wherein the pancreatic stimulation is responsive to the vagus nerve to slow heart rate and secrete insulin.

9. The method of claim 1, wherein the Pancreatic stimulation therapy does not cause hypoglycemia

10. The method of claim 1, wherein the Pancreatic stimulation therapy does not cause any long term side effects as caused by modern medicine

11. The method of claim 1, wherein the pancreatic stimulation therapy improves cognitive functions.

12. The method of claim 1, wherein the Pancreatic stimulation Improves the functions of the beta cells to provide better blood sugar regulation

13. The method of claim 2, wherein the pancreatic stimulation reduces a risk of myocardial infarction.

14. The method of claim 2, wherein the pancreatic stimulation therapy reduces a risk of sudden cardiac complications.

15. The method of claim 2, wherein the pancreatic stimulation reduce a workload of a diabetic heart.

16. The method of claim 2, wherein the pancreatic stimulation therapy is applied to reduce ventricular contractility of a diabetic heart.

17. The method of claim 1, wherein the external pancreatic stimulation therapy is adapted to reduce norepinephrine release to attenuate ventricular remodeling of a diabetic heart.

18. The method of claim 1, wherein the pancreatic stimulation is adapted to lower arterial peripheral resistance.

19. The system and method of claim 1, wherein the pancreatic stimulation is adapted to insulin release to promote glucose uptake and metabolism.

20. The method of claim 1, wherein the pancreatic stimulation enhances a parasympathetic response to reduce the workload of the diabetic heart.

21. The method of claim 2, wherein the diabetic patient has poor glucose control and is experiencing risk of myocardial infarction, applying the pancreatic stimulation reducing a risk of diabetic complication

22. The method of claim 1, wherein pancreatic stimulation reduces the workload of the diabetic kidney includes delivering stimulation to elicit a parasympathetic response, including sympathetic nerve activity or delivering stimulation to inhibit sympathetic nerve activity.

23. The method of claim 2, wherein administering a diabetes treatment comprises of engaging in focused exercise.

24. The method of claim 2, wherein said pre-determined amount of pancreatic stimulation is administered daily.

25. The method of claim 2, wherein said safe, pre-determined frequency of pancreatic stimulation comprises at least twice daily on an empty stomach.

26. The method of claim2, wherein said safe, pre-determined duration of stimulation comprises at least daily as and when needed.

27. The method of claim 2, wherein the method is designed to treat obesity and Type II diabetes.

28. The method of claim 2, wherein the treatment of conditions is comprised of regulating blood sugar levels or regulating insulin production.

29. The method of claim 2, wherein the pancreatic stimulation increases the blood supply and oxygen supply that in return improves efficiency and the functioning of beta cells.

30. The method of claim 1, wherein the diabetic patient has poor glucose control and is experiencing the period of low physical exertion includes delivering stimulation to secrete insulin, delivering stimulation to reduce ventricular contractility, delivering stimulation to reduce norepinephrine release to attenuate ventricular remodeling, delivering stimulation to induce coronary artery vasodilatation, delivering stimulation to lower arterial peripheral resistance, or delivering stimulation to stimulate insulin release to promote glucose uptake and metabolism, delivering stimulation to reduce weight, delivering stimulation to reduce blood glucose levels of a diabetic.
